(19) United States
(12) Patent Application Publication
(10) Pub. No.: US 2017/0196202 A1
(43) Pub. Date: Jul. 13, 2017

(54) METHOD AND PRODUCT FOR MANAGING PET CARE

(71) Applicants: PETER RAGONETTI, Brooklyn, NY (US); SHAHIR AHMED, Denver, CO (US)

(72) Inventors: PETER RAGONETTI, Brooklyn, NY (US); SHAHIR AHMED, Denver, CO (US)

(21) Appl. No.: 15/316,487

(22) PCT Filed: Jun. 5, 2015
(86) PCT No.: PCT/US15/34517

§ 371 Date: Dec. 5, 2016

Related U.S. Application Data
(60) Provisional application No. 62/008,857, filed on Jun. 6, 2014.

(51) Int. Cl. A01K 29/00; A61B 5/00; A61B 5/11; A01K 11/00; A01K 15/02

(52) U.S. Cl. CPC A01K 29/005; A01K 11/008; A01K 15/021; A61B 5/1118; A61B 5/7475; A61B 5/0022; A61B 2562/0219; A61B 2503/40; G06F 19/3418

(57) ABSTRACT

A method and product for collecting, recording, transmitting, and managing data associated with pet care and using the data to create an improved and more engaging pet owning experience.

### CROSS-REFERENCE TO RELATED APPLICATIONS

This application claims priority pursuant to 35 U.S.C. 119(e) to co-pending U.S. Provisional Patent Application Ser. No. 62/008,857, filed Jun. 6, 2014, the entire disclosure of which is incorporated herein by reference.

### FIELD OF THE INVENTION

The present invention relates to a method and product for collecting, recording, transmitting, and managing data associated with pet care and using the data to create an improved and more engaging pet owning experience. Specifically, the invention comprises a pet tag with a plurality of buttons that can communicate wirelessly with a smart phone, tablet, other computer devices, computer networks, or wireless telecommunications networks to collect, record, transmit, and maintain information about a pet's care and activities through a specially designed application.

### BACKGROUND OF INVENTION

The use of electronic or digital pet tags is generally known in the art for use in identifying a pet or the pet's owner, for tracking the location of a pet, or for allowing a pet to access food or shelter. For example, U.S. patent application Ser. No. 13/456,173 describes a digital pet tag for storing and displaying identification data for a pet. U.S. patent application Ser. No. 11/386,281 describes digital pet information storage and identification device. U.S. patent application Ser. 13/894,100 describes an animal collar or tag that can communicate by radio frequency with a base unit to identify and train pets from bad behavior such as barking. U.S. Pat. No. 7,055,463 describes and claims a method for web-based pet registration and retrieval system associated with a rabies tag. The expressed purpose of the '463 Patent is to locate owners of lost pets. U.S. patent application Ser. No. 13/889,652 describes the use of a pet collar for tracking movement and locations of pets. U.S. Pat. No. 7,685,966 describes and claims the use of an electronic pet tag to provide access to a lidded pet dish. U.S. Pat. No. 8,947,240 describes and claims an animal data collection system.

This prior art does not disclose or claim the use of an electronic pet tag wirelessly connected to a smart phone or other computer device for purposes of recording and maintaining pet activity or interaction with a pet as described in the present invention.

It would be desirable to provide a product and method that allow a pet owner or pet caretaker to record, maintain, and monitor pet activity and pet interaction such as walking, feeding, and providing health care through a wireless connection with the owner's smart phone, tablet or other computer device.

### SUMMARY OF THE INVENTION

The present invention relates to a method and product for collecting, recording, transmitting, and managing data relating to pet care, training, and interaction and allowing the collected data to be used and manipulated for improving the care and training of the pet and the pet owner's interaction of the pet.

One object of the invention is to provide a plurality of means for collecting and recording pet related activities, such as play, walking, feeding, and medication. In a preferred embodiment, the invention includes an electronic pet tag or orb that has a plurality of buttons and is coupleable to the pet. The pet tag includes components that allow for certain actions or activities to be detected and recorded. By way of example, the pet tag in a preferred embodiment includes an accelerometer that will detect pet activity or inactivity and a means for recording the activity or inactivity. The pet tag may also contain a global positioning system (GPS) to track and monitor a pet's location. In one embodiment, the accelerometer and GPS may be activated or deactivated by pressing one or more buttons on the pet tag.

The plurality of buttons on the pet tag are pre-programed singularly or in combination to associate with certain activities, such as feeding, walking, training, and mediations. The pressing of a button or buttons causes the activity to be recorded and maintained on the pet tag. The buttons may also be pre-programed to activate or deactivate other functionality of the pet tag such as accelerometer or GPS. This will reduce power usage and allow the owner to select when to use such functions.

Another object of the invention is to provide for wireless communication of the pet tag to a specially designed software application loaded on a smart phone, tablet, other mobile device, or other computer device. In the preferred embodiment, the pet tag will include Blue Tooth and Wi-Fi components to allow for wireless connectivity to a variety of computer devices and to wireless computer networks. The application allows the pet owner or caretaker to create profiles for a pet and to collect, record, maintain, and manage data associated with a pet's activities and the pet owner's interaction with the pet. When the pet tag is in communication with the computer device with the loaded application, the data and information collected and recorded on the pet tag is communicated to device and compiled within the application. The application also allows pet activities to be entered on the mobile or computer device directly.

Another object of the invention is for the pet tag to communicate wirelessly with a plurality of computer devices for example where there are multiple pet owners or where pet care is provided by a third party. The data collected on the plurality of devices will be compiled within the pet's profile and assessable remotely to allow pet owners to monitor the pet's activities.

A further object of the invention is to provide an application that utilizes the data collected from pet activity to help improve or modify pet care or pet training for the benefit of the pet and to provide a more rewarding pet ownership experience.

### DESCRIPTION OF DRAWINGS

The accompanying figures are incorporated herein and form a part of the specification for the present invention and further illustrate the present invention:

FIG. 1 is a front view of one embodiment of a pet tag of the present invention showing a plurality of buttons for registering pet actions or interaction.

FIG. 2 is a perspective view of one embodiment of a pet tag of the present invention showing a plurality of buttons for registering pet actions or interaction.

FIG. 3 is a side view of one embodiment of a pet tag of the present invention showing a plurality of buttons for registering pet actions or interaction.

FIG. 4 is a back view of one embodiment of a pet tag of the present invention showing a plurality of buttons for registering pet actions or interaction.

FIG. 5 is an exploded view of one embodiment of a pet tag of the present invention showing a plurality of buttons for registering pet actions or interaction.

FIG. 6 is a front view of a second embodiment of a pet tag of the present invention showing a plurality of buttons for registering pet actions or interaction.

FIG. 7 is a rear view of a second embodiment of a pet tag of the present invention showing a plurality of buttons for registering pet actions or interaction.

FIG. 8 is an illustration a pet tag of the present invention wirelessly connected to a computer device with the pet tag application.

FIG. 9 is an illustration of the method of the present invention showing the relationship between the pet, the pet tag, the computer device, and the application.

FIG. 10 is an additional illustration of the method of the present invention showing the relationship between the pet, the pet tag, the computer device, and the application.

FIG. 11 is a flow diagram showing examples of the method of the present invention.

### DETAILED DESCRIPTION OF THE INVENTION

The present invention is best understood by reference to the drawings and description set forth herein. Embodiments of the invention are discussed below with reference to the drawings. However, those skilled in the art will readily appreciate that the detailed description given herein with respect to the drawings is for explanatory purposes as the invention extends beyond the limited embodiments described. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention.

The present invention should not be limited to the particular embodiment, methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means. Also, singular words should be read as plural and vice versa and masculine as feminine and vice versa, where appropriate, and alternative embodiments do not necessarily imply that the two are mutually exclusive.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number for a claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations. However, the use of such phrases should not be construed to imply a claim recitation by the indefinite articles “a” or “an” limits any particular claim containing such claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of claim recitations is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

Throughout this disclosure, examples will be provided for the method and product of the present invention. However, those of skill in the art will appreciate additional applications for the disclosed method and product.

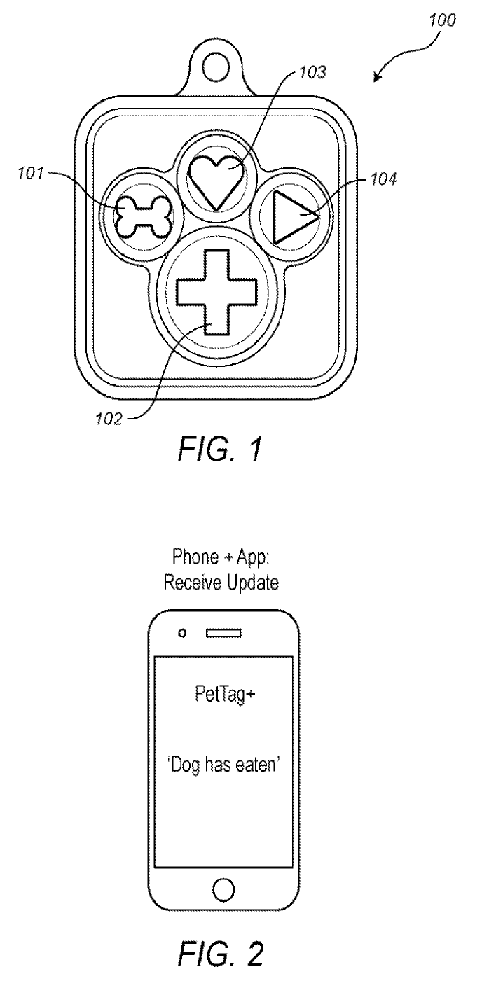

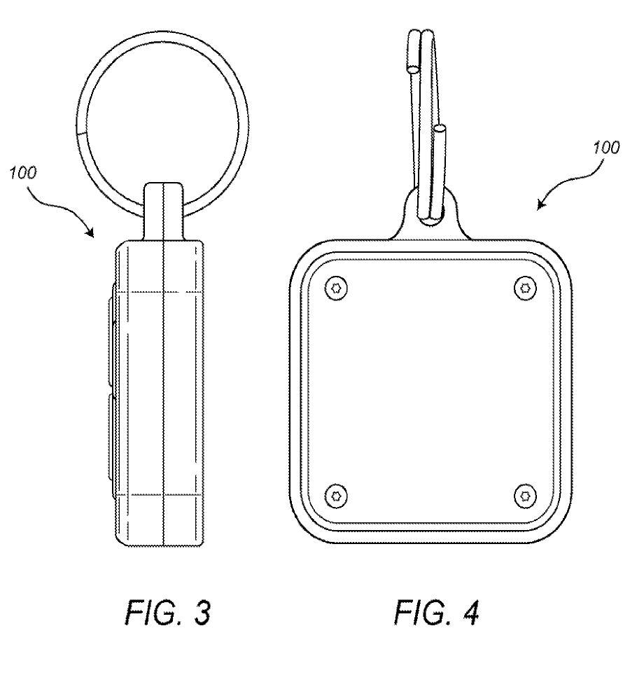

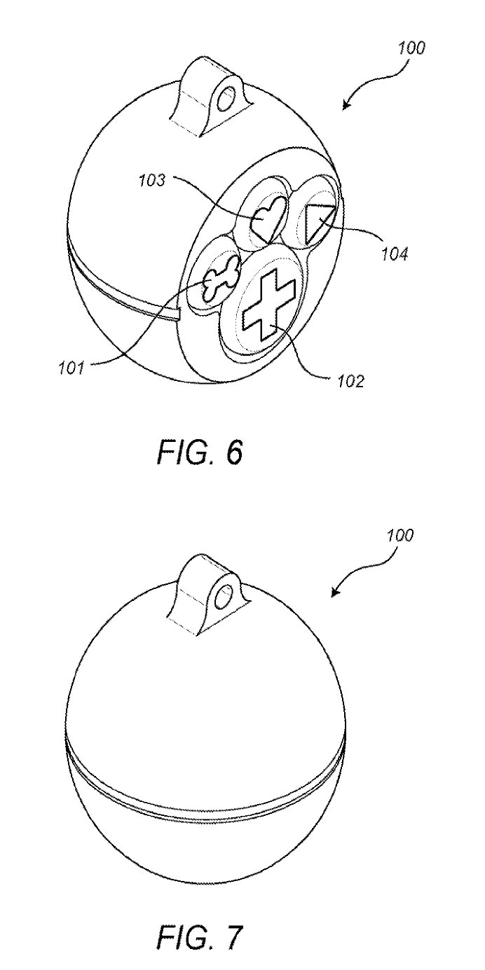

Embodiments of the pet tag 100 of the present invention are provided in FIGS. 1 to 7. In each of these embodiments, the pet tag 100 has four separate buttons 101 to 104. Each button 101 to 104 or combination of buttons 101 to 104 on the pet tag 100 is associated with certain actions or activities of the pet, the pet owner, or pet caretaker, for example feeding, walking, play, providing affection, and a checkin/checkout function. A pet tag 100 may have more or fewer than four buttons 101 to 104.

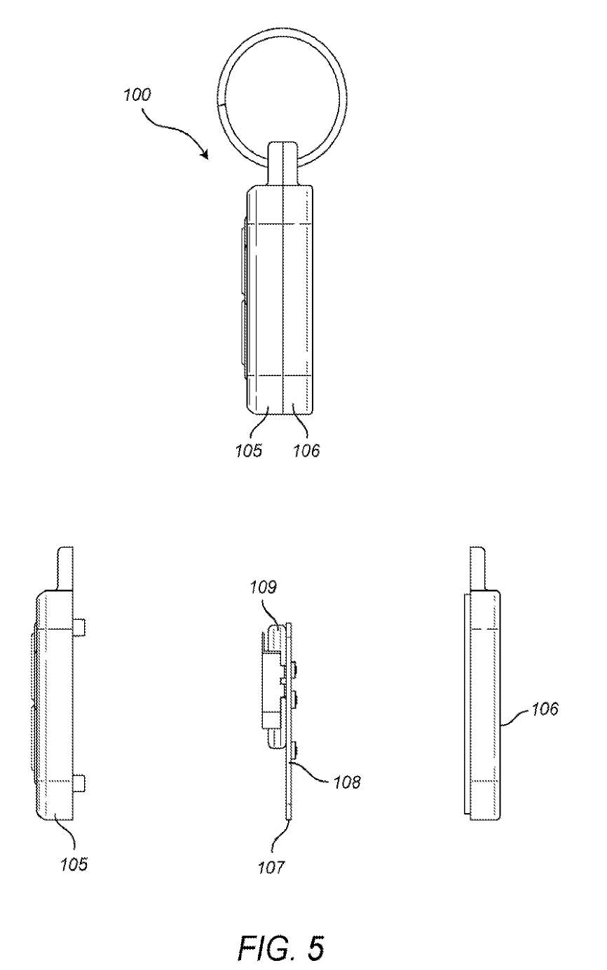

In a preferred embodiment of the pet tag 100 as shown in FIG. 5, the pet tag 100 has a front housing 105 with four buttons 101 to 104 on the face of the front housing 105, a back housing 106 that is removably attached to the front housing 105, and internal electronic components 107 comprising a circuit board 108 and a battery 109. In a preferred embodiment, the circuit board includes a computer chip, a memory chip, wireless communications capability, such as Blue Tooth, WiFi or other wireless technology, an accelerometer, and GPS. An accelerometer can be used to detect pet activity or inactivity. GPS can be used to identify and monitor a pet's location. In certain embodiments, the accelerometer and GPS, as well as other features of the pet tag 100 may be activated or deactivated using one or more of the plurality of buttons 101 to 104 of the pet tag 100.

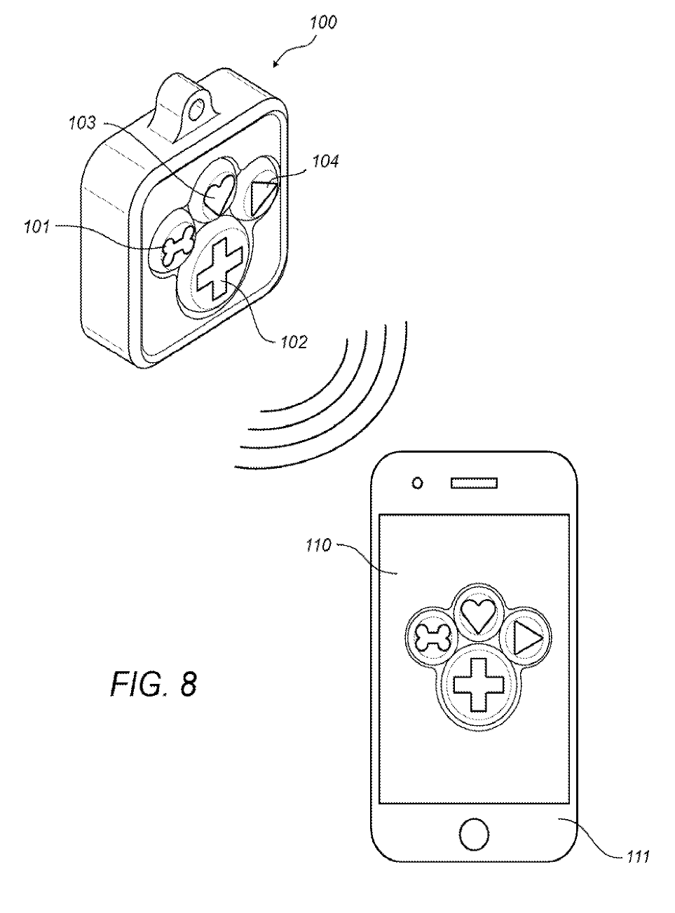

As illustrated in FIG. 8, a preferred embodiment of the method of this invention is the pet tag 100 connected via Bluetooth or other wireless technology to an application 110 that is loaded on a smart phone, tablet, or other mobile or computer device 111. The circuit board 108 for the pet tag 100 is configured so that the pet tag 100 connects to the application 110 when any button 101 to 104 on the pet tag 100 is pressed or activated. Therefore, by pressing a button 101 to 104 or a series of the buttons 101 to 104 a pet owner or caretaker can record and maintain certain activity of the pet or interactions between the pet and the owner or caretaker. The buttons 101 to 104 may also be used to activate or deactivate other features of the pet tag 100 such as the accelerometer or GPS.

In a preferred embodiment, the application 110 is specially designed to provide controlled and secure communication with the pet tag 100 and includes features and functions such as: (a) a pet profile page where a pet owner can create and maintain a pet profile; (b) fields that are populated with data communicated from the pet tag 100; (c) the ability to record pet activities directly on the computer device 111 using the application 110; (d) the ability to compile and assimilate pet activity and compare the activity of the pet to preferred pet activities for the pet and suggest improvements or modifications to pet care or activity; and (e) the ability to share pet activity with a circle of pet friends or caretakers who have the application 110 loaded on a computer device and access to the pet profile. This last function allows a pet owner for example to access pet data when the pet is at day care or veterinarian.

Once the pet tag 100 is wirelessly connected to the application 110 on the computer device 111 and the application 110 remains open on the computer device 111, the user may record pet activity by pressing the appropriate button 101 to 104 on the pet tag 100, causing the activity to read on the computer device 111. This allows the user to record pet activity and actions on a computer device 111 without entering the data and information directly in the computer device 111. In one embodiment, pressing a button 101 to 104 on the pet tag 100 with the computer device 111 in range will open the application 110 and the activity associated with the button 101 to 104 being pressed on the pet tag 100 will be registered to the profile for the pet in the application 110.

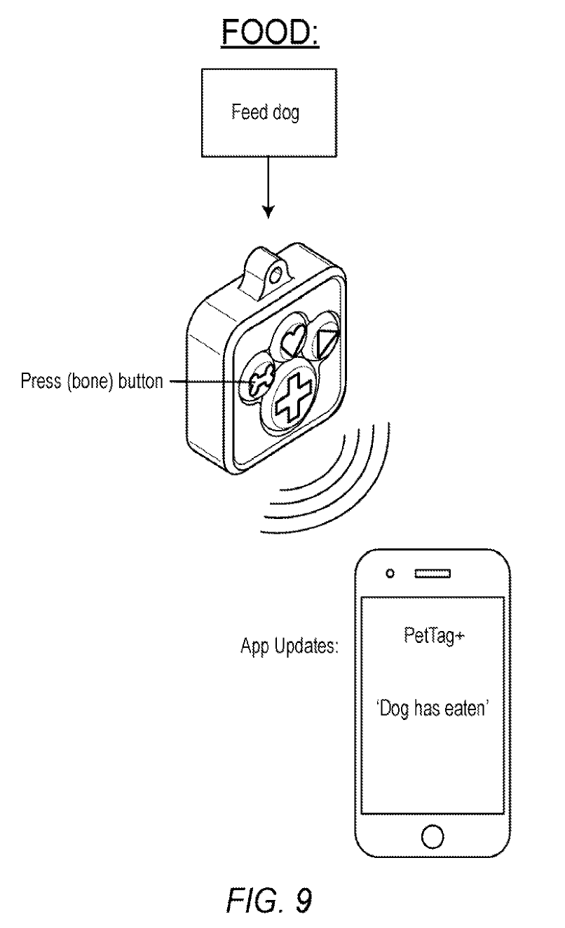

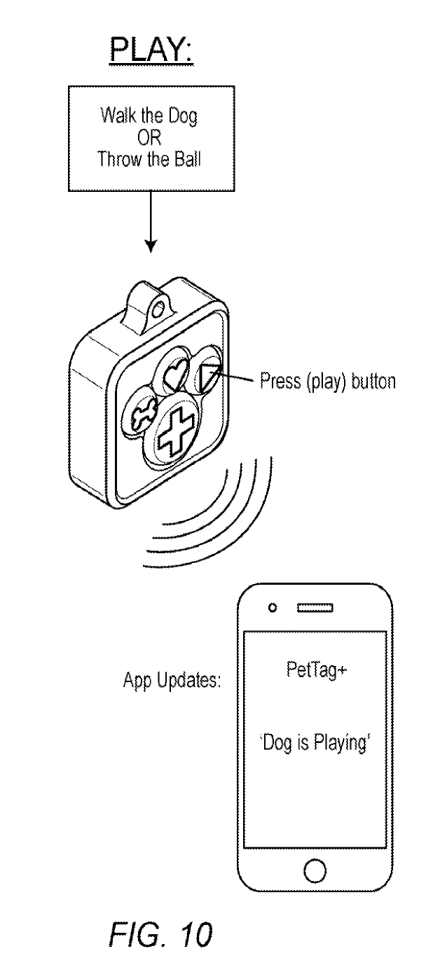

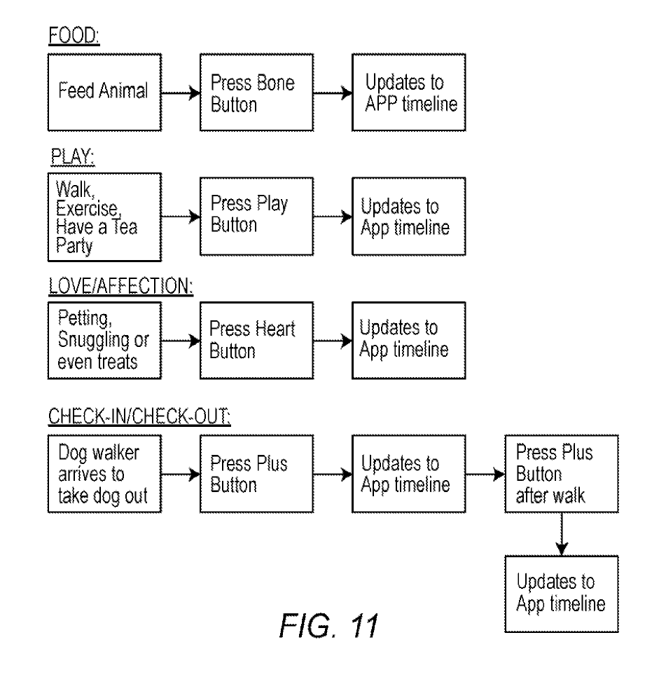

Examples of the method for managing pet care are set forth in FIGS. 9 and 10. In these examples, the pet tag 100 contains four buttons 101 to 104, a button with a bone symbol 101, a button with a heart symbol 103, a button with a triangular play shape 104, and a button with a plus sign 102. Each button or combination of buttons 101 to 104 on the pet tag 100 is associated with certain actions or activities, for example feeding, walking, play, providing affection, and a checkin/checkout function.

In one embodiment of the method as set forth in FIG. 9, when the pet is fed the user can press the “bone” button 101, which sends information to the application 110 that updates the pet profile to indicate that the pet was fed and the time of the feeding. FIG. 10 shows the method for recording play of the pet using the “play” button 104, which can be used to record walks, exercise, or general play with the pet. The “heart” button 103 can be used to record general affection, medication, or other treatments. The “plus” button 102 is used to checkin and checkout of the longer activities, like a dog walker checking in to walk the animal and out when the walk is finished, this is performed with a combination of button 101 to 104 presses.

The buttons 101 to 104 can also be pressed in predetermined combinations to trigger other actions in the application 110. For example long walks can be recorded by the user pressing the play button 103 and then the plus button 102 to register the time the walk began. Then at the end of the walk, the user can press the play button 103 and plus button 102 to register the time the walk ended. Pressing a button combination in this matter results in different data being collected and stored by the application 110. Programed button combinations can also be used to initiate other programs in application 110 for training or pet games.

The pet tag 100 of a preferred embodiment may also collect and record data automatically relating to pet activity or inactivity by using an accelerometer, GPS, or other similar devices. Data collected by the pet tag 100 can be transmitted and recorded on a computer device 111 using the application 110. In one embodiment, the accelerometer, GPS and other devices may be activated or deactivated by pressing one or more buttons 101 to 104 of the pet tag 100.

In a preferred embodiment, data collected from the pet tag 100 is stored in the animal profile on the application 110 in timeline so a user can ensure that all basic needs for the pet are being met. The application 110 may also provide push notifications to all individual computer devices 111 that have access to the pet profile, allowing multiple owners or caretakers to monitor a pet's needs and activities. For example, a notice is sent when the pet is to be fed and another notice is sent when the pet has been fed. This allows a pet owner to know that the pet is receiving the proper care and attention even when the owner is absent or unable to provide the care directly. The notification feather also prevents overfeeding or overmedication of a pet that may have more than one owner.

In other embodiments, the method of the present invention includes pet products that are wirelessly connectable to the pet tag 100 that allows the activity to be collected and maintained on the pet tag 100 and then communicated to the application 110 on a computer device 111. By way of example, a feeding bowl may contain electronics that communicate wirelessly with the pet tag 100 and record data about when a pet has eaten and the amounts of food the pet has consumed. Other examples would be toys containing electronics that communicate wirelessly with the pet tag 100 and record data about when, how often, and how extensively a pet plays with the toy.

It should be understood, of course, that the foregoing relate to exemplary embodiments of the invention and that modifications may be made without departing from the spirit and scope of the invention as set forth in the following claims.

What is claimed is:

1. A product for managing pet care comprising
a tag with a plurality of buttons and a specially designed software application loaded on a computer device wherein
said tag is removably attachable to a pet, has a computer chip, a power source, a means for wirelessly communicating with said specially designed software application, and a memory chip;
said tag collects and records data concerning pet activities by activating one or more of the plurality of buttons; and
said tag wirelessly communicates the data to the specially designed software application loaded on a computer device.

2. A product of claim 1 wherein the tag communicates wirelessly with one or more other pet products to record pet activity in relation to said other pet products.

3. A product of claim 1 wherein the tag contains an accelerometer to detect pet activity.

4. A product of claim 1 wherein the tag contains GPS to identify the location of a pet.

5. A product of claim 1 wherein the specially designed software application is loaded on one of a smart phone or tablet.

6. A product of claim 1 wherein pet activity is recorded by entering the activity on the computer device where the specially designed software application is loaded.

7. A product of claim 3 wherein the GPS is activated by pressing one or more of the plurality of buttons.

8. A product of claim 4 wherein the accelerometer is activated by pressing one or more of the plurality of buttons.

9. A method for managing pet care comprising:
a specially designed software application;
a tag with a plurality of buttons, a computer chip, a power source, a means for wirelessly communicating with said specially designed software application, and a memory chip;
loading the specially designed software application on a computer device;
removably attaching the tag to a pet;
collecting and recording data about activities of the pet by activating one or more of the plurality of buttons on the tag; and
wirelessly communicating the collected data to the specially designed software application.

10. A method for managing pet care comprising:
a specially designed software application;
a tag with a plurality of buttons, a computer chip, a power source, a means for wirelessly communicating with said specially designed software application, and a memory chip;
loading the specially designed software application on a plurality of computer devices;
removably attaching the tag to a pet;
collecting and recording data about activities of the pet by activating one or more of the plurality of buttons on the tag; and
wirelessly communicating the collected data to the specially designed software application loaded on the plurality of computer devices.
